(19) United States
(12) Patent Application Publication
(10) Pub. No.: US 2012/0053603 A1
(43) Pub. Date: Mar. 1, 2012

(54) WOUND CLOSURE CLIPS, SYSTEMS AND METHODS

(75) Inventor: Warren P. Williamson, IV, Loveland, OH (US)

(73) Assignee: CLEVEX, INC., Columbus, OH (US)

(21) Appl. No.: 13/319,162

(22) PCT Filed: May 7, 2010
(86) PCT No.: PCT/US10/34033

§ 371 Date: Nov. 7, 2011

Related U.S. Application Data
(60) Provisional application No. 61/176,233, filed on May 7, 2009.

(51) Int. Cl. A61B 17/03; C23F 1/00

(52) U.S. Cl. 606/151; 216/11

(57) ABSTRACT

Wound closing clips (70, 90) each include a clip body (70a, 90a) and a first plurality of teeth (74, 94) extending in a first direction from the clip body (70a, 90a) for engaging superficial skin tissue. A second plurality of teeth (76,96) extends in a second direction from the clip body for engaging tissue below the superficial tissue, the second direction being different than the first direction and configured to direct the second plurality of teeth into deeper tissue than the first plurality of teeth and a flexible tensile member connecting portion coupled with said first and second pluralities of teeth (74, 76, 94, 96). The flexible tensile member connecting portion (82, 84, 92, 93) includes an element capable of securing a flexible tensile member (62) extending below the tissue engaged with the second plurality of teeth (76, 96) and across a base of the wound (50) resulting in a wound closing system.

This application claims the priority of U.S. Provisional Patent Application Ser. No. 61/176,233 filed on May 7, 2009 (pending), the disclosure of which is incorporated by reference herein.

### TECHNICAL FIELD

This invention generally relates to the surgical approximation of skin.

### BACKGROUND

Skin biopsy is one of the most important diagnostic tests for skin disorders. Skin biopsy procedures are performed in many ways. If an improper technique is used to remove a lesion and it turns out to be malignant, proper diagnosis of the stage of cancer may be impossible. However, once a pathologist has established the presence of malignant cells a second surgery is almost always performed to make certain all remnants of the cancerous tissue have been removed; this procedure is called a full depth excisional biopsy with margin. As such, these procedures must take out large portions of tissue to be certain that not only are the malignant cells removed but that there is sufficient clear margin of healthy tissue surrounding the malignant tissue. The margin and diagnosis must be confirmed through histopathology preparation of the biopsy sample and cross-sections of the excised tissue reviewed by a pathologist. Therefore, in contrast to a simple biopsy procedure which is more exploratory than curative in nature, a full depth excision is much more invasive and leaves a larger and deeper wound to close.

There are many techniques and surgical tools that can be used to remove a tissue sample for diagnosis. Most commonly a scalpel is used to make a surgical incision, and a hemostatic device, such as a cautery tool or clotting agent, is used to stop the bleeding while removing the tissue in question and approximating the severed edges to close the wound for healing. See reference document: Fusiform Excision by Thomas J. Zuber M.D. In the prior art there have been attempts to provide mechanically enhanced wound closure techniques, but most are as time consuming as suturing itself. In addition with current techniques, consistent, effective control of the tissue is difficult. Over or under penetration of the skin engaging portions of wound closure devices can be detrimental or make it impossible to consistently control the wound edges. Devices that employ slits in the skin (into which mechanical holding legs are inserted) without some way to engage the dermis will likely fail to keep the wound approximated and result in slippage of the wound edges or worse.

Often, a skin lesion biopsy results in an opening that is much larger than can be comfortably closed by previously disclosed skin clip devices. When full depth excisions get to be larger than about 12 mm it is recommended that the surgeon use a layered wound closure technique. Layered closures include a subcutaneous set of interrupted sutures to approximate the base layers of the wound, with a second set of cutaneous or surface layer sutures to approximate the epithelial layer of the skin. If the surgeon does not use a layered closure there is a high probability that a hollow pocket will form below the skin surface. If such a pocket were to form it would quickly fill with blood or thrombus, which turns into a clot that is then replaced by cellular structures which form a scar. To create an appealing cosmetic wound closure it is important to prevent any excess of clotted material from forming below the skin surface. A layered closure solves this problem by approximating the lower layers of the skin tissue directly above the fat. These layers are known as the reticular dermis.

Over time the byproducts from healing can erupt to the skin surface exposing the shiny collagen structure seen in a large scar. Unless the patient is in a combat or trauma situation surgeons for the most part try to make a cosmetically appealing wound closure. Plastic surgeons are taught special techniques to make especially aesthetic healing lines. However these are time-consuming and tedious procedures.

Typically, when a skin lesion is removed for biopsy analysis, the resulting wound is closed to promote quick healing with a minimum of scar tissue formation. The wound is closed by various methods, most often with sutures or adhesive strips. While small wounds in the flesh are able to be closed with a single surface layer of sutures, a layered closure is required for a size threshold at around 10-12 mm in length, or when the skin has been fully penetrated and is bleeding profusely. A layered closure requires that a primary set of sutures be placed deep in the wound base to approximate the subcutaneous layer of tissue. By having a deep set of sutures in the wound base, the high tensional forces required to rejoin the tissue are buttressed deeply at the fascial layer making up for the material that was removed. This prevents “pocketing” where a large clot can form in the pocket. A pocketed wound can result in a weak or infected wound. The deeper tissue layers heal more completely when brought into close approximation during healing. This also allows for a more delicate suturing of the thin epithelial layers to minimize the scar. This is important when making a cosmetic repair of the skin since the skin reacts to stretching at the entry point to a suture. Concentrated stretching will produce a collagen reaction which shows up as a shiny, light colored scar. This is why tiny tracks are seen in an interrupted suture line.

Sometimes a practitioner may try to close the wound with a single line of interrupted sutures when a double layer would be indicated. An undesirable scar may result after suturing a wound without a layered closure. The use of only a single line of sutures may result in a wound closure that alternates between being too tight and too loose. To combat this, surgeons typically place sutures more closely together than are required for healing, and over-tighten sutures to make sure that as the swelling is reduced around the incision the wound will not dehisce or open up. Overly tightened sutures stretch the skin and the skin reacts to this trauma by making scar tissue around the stretched area (i.e., the suture exit point) which results in the “tracks” seen across poorly sutured wounds.

The choice of suture materials also plays an integral role in the cosmesis of wound healing. A surgeon concerned with the appearance of the final scar will use a monofilament for the top layer approximation. Monofilament is known as a nonreactive suture material. However it is far more expensive than the standard materials. Monofilament is also non-absorbable, therefore it is never used for the buried base layer sutures.

Another procedural complication arises when more than one buried suture is required. For instance, if three buried sutures are required to close a wound the surgeon will not be able to complete each buried suture in series, because as he approximates the base layers the wound continues to close up. It then takes an enormous amount of skill and dexterity to place the next base suture needle correctly because the wound closes and the surgeon does not have access to the proper layers of tissue. Therefore, one skilled in this procedure will use many separate sutures and place all of the sutures in the base first while leaving their loose ends splayed above the wound. Then the surgeon will tie each set of knots and drive the knots down below the skin level into the lowest layers of the wound, in series, cinching up the base layer tissue along the way. It is much easier to drive a knot deep into the wound than it is to gain visual access to the proper layers to place the needles after the wound closes from the adjacent base layer suture. Exact placement of the entry point of the needle will determine whether or not the layers of skin approximate evenly when they are cinched closed. Therefore, misplacement of the needle due to compromised visual access will result in a wavy or uneven healed scar. While this technique is taught and practiced every day, it requires a new suture needle combination for each base layer. Sometimes the doctor can use one half of the length of the suture for the first placement, then cut it in half for a second placement. Proper technique for suturing requires at least 6 to 8 inches of free suture length to be able to tie the knots with gloves on. Therefore, this technique requires many suture packages for a single wound closure resulting in an inefficient and expensive use of suture, not to mention a time-consuming procedure for the doctor or surgeon. As mentioned previously, the base layer sutures are made of an absorbable material either natural (gut) or synthetic. The body must be able to break down this material to absorb it. The absorption process leaves behind scar material in the wound.

Another important aspect to consider when suturing large wounds is the effect of edema or swelling of the surrounding tissues. Whenever trauma to the skin occurs, natural vasoconstriction results as the body tries to prevent blood loss. In addition to the natural traumatic tissue responses most local anesthetics, like Lidocaine, which are used to numb the pain sensation at the incision site, also contain a vasoconstrictive agent such as epinepherine to help staunch bleeding at the incision site. Arterial side pressure builds up fluid at the trauma site with no venous outlet and so edema results. This resultant edema makes it impossible to have the suture at the same proper tightness initially, during the wound closure and after the swelling has reduced the bulk or fullness of the tissue. This means that the surgeon is taught to “over-tighten” the suture knots to make up for the slackening of the sutures as the swelling subsides (so that the wound will not reopen after about 24 hours). The looseness of the skin once the swelling has been reduced can cause the suture line to open and the wound to dehisce. This can leave a large gap to fill with scar tissue and may be susceptible to infection. Therefore, it is very difficult to pre-tension the suture properly so that it will not be too tight during the vasoconstriction period or too loose once the edema has resolved. The problem with sutures that are too tight is that the skin stretching that occurs at the suture exit points creates scars. Skin reacts to stretching very quickly (evidenced in the common, permanent stretch marks that pregnant women can develop). In the case of wound closures, the point of highest tension is at the suture exit points, therefore elongated (stretched) holes or tracks at the suture exit can develop.

Three elements of good wound closure therefore have been:

1) Deep approximation of tissue to prevent pocketing combined with fine surface sutures and proper skin tension by surface sutures.

2) Proper wound tension throughout the healing process to keep the wound edges together after the edema has subsided.

3) Precise alignment of the tissue layers for cosmetic improvement of the scar.

### SUMMARY

In one illustrative embodiment, a wound closure system is provided that produces the same closure effect as a layered suture technique by combining the approximation of the deep base layer with a fine controlled surface layer approximation. Stated another way, such an embodiment provides both a deep approximating flexible tensile member such as a suture, and a surface tension clip. Each clip is capable of engaging the thinner upper layers of the skin and spreading the approximation forces or load. By spreading out the approximation forces, the skin is subjected to less localized stretching trauma which results in finer or nonexistent “tracks” adjacent to the wound edges. In addition, by incorporating a spring element or compliant tensioning device into the system, close approximation of the tissue edges is assured throughout the healing process, despite the changing hemostasis and edema of the surrounding tissues. Sutures used in various embodiments herein can be removed at the end of the procedure, including the suture(s) in the deep penetrating layer. Therefore, a monofilament suture can be used, taking advantage of its less reactive nature even in the deep base layering step.

In one embodiment, the closure mechanism of the invention comprises two separate elements—a flexible tensile member such as a suture, and a set of skin-engaging, wound-tensioning clips. The suture is placed deeply from a first surface adjacent the wound across the wound base and out through a second surface adjacent the wound. The set of skin clips deploy into skin surfaces adjacent the wound neatly approximating the skin surface when the suture ends are secured into the clips.

One or more embodiments of this invention enhance the skin penetrating attributes of the wound tensioning clips by controlling the skin edges with tines or teeth which engage the skin. The tooth should be sharp enough to initially penetrate the epidermis to fix the tooth into the skin. It takes a rather small sharp point to penetrate the skin, however if the tooth profile is too long and slender the tooth can either bend or over-penetrate. Over-penetration is a condition in which the depth of the penetrating tip is not controlled. This will cause a situation where the tooth continues to penetrate into the tissue during the normal course of skin stretching and relaxation during the healing process. In one embodiment a novel tooth profile disclosed herein solves this problem by having a stepped profile. It combines both an initial sharp penetrating point and a compression stop element which controls the penetrating depth. By combined use of the sharpness undercut depth and included angle of the penetrating point and the secondary compression stop element, the clip can both penetrate and compress the wound edges at each penetration point. The compression point or stop element is equally as important as the penetration control. As the initial penetration stops at the compression stop element, the further movement of the clip and compression of the tissue urges the edges of the incision together in a very controlled manner.

Interestingly, even a suture cannot perform as a system according to this invention can because the suture maintains its diameter along its entire length. The penetration is of course provided by the needle and wound compression is controlled by the tightness of the knots. However, the force vector for gathering the tissue with a traditional suture that has been pulled over top of the wound towards the incision creates undue stress and stretching of the tissue. This creates scar tracks at the suture exit wounds. The profile of teeth formed according to one embodiment of this invention combines both features (penetration and compression) but again in a way that is more controllable than through standard suturing.

Using standard suturing techniques, the point of entry for the suture is determined by hand. Therefore, if the first and second suture needle puncture points being placed across opposite sides of a wound are not perfectly aligned both laterally along the incision and spaced back from the incision edge, a mismatch will occur when the two sides are approximated with the knot. In an embodiment of the disclosed system a long row of teeth each with stepped tissue penetrating and gathering features assures uniform placement and gathering of the tissue edges. When the two sides of the wound are brought together the alignment between the skin edges is assured. In addition since a plurality of teeth are aligned on each side of the wound a straight tooth penetrating line is assured on each edge. Bringing these two straight lines together allows the compressed epidermis to evert and rollback on the top side of each penetrating clip. This creates a very positive condition for healing and minimized scar production. As the wound heals the tissue above the clip sloughs off and dies. The tissue below the clip remains patent and the wound heals with minimum scar production.

It should be noted here that this invention will be of use in many areas including, but not limited to, skin lesion removal and closure of the resulting wound, skin grafts, plastic surgery, and general repair of surgical incisions or other wounds in the skin. For example, this invention will be applicable to any procedure or anatomical skin incision where a multilayer suture technique is used today. In the body of this disclosure, the terms incision and wound are used interchangeably to refer to the opened area in the skin which needs to be surgically closed.

Various additional features, aspects and methods of wound closure will be more readily appreciated upon review of the following detailed description of the illustrative embodiments taken in conjunction with the accompanying drawings.

### BRIEF DESCRIPTION OF THE DRAWINGS

FIG. 1 is a schematic perspective view of a wound being closed with the prior art technique using deep-buried, interrupted sutures.

FIG. 2 is a schematic perspective view of the serial closure of the prior art deep-buried sutures with knots that will reside subcutaneously.

FIG. 2A is a side cross section illustrating the subcutaneous knots of the prior art below the surface of the skin with the resulting bump at the surface of the healed wound.

FIG. 3 is an exaggerated top view of interrupted surface sutures of the prior art, showing the suture exit areas which have been over-tightened to account for the eventual resolution of traumatic edema, separated by areas where the tissue can relax and open between each suture without any mechanical support.

FIG. 3A is a top view showing the stretched hole which results at the suture exit site in the prior art because the suture must initially be over-tightened to account for the eventual resolution of traumatic edema.

FIG. 4 is a cross sectional view of a typical wound.

FIG. 5 illustrates a needle and suture being passed from the first side of the incision or wound of FIG. 4 through the deep base and out through the second side of the incision during application of a clip according to an embodiment of the invention.

FIG. 6 shows the first skin clip according to an embodiment of the invention, put in place in the first side of the wound with clip placement tool.

FIG. 7 shows the cross-section of the wound where the first clip is in place and either the knot in the suture is pulled against the knot stop feature or the suture itself is pulled into the pinch groove to hold the suture fast.

FIG. 8 illustrates a second clip introduced on the second side of the wound with the clip placement tool.

FIG. 9 illustrates the final tensioning step with the two clips of the clip set on opposite sides of the wound.

FIG. 10 illustrates the first suture tail crossing over the wound opening and placed under one of the cleats on the second clip to pull the top of the wound edges together.

FIG. 11 shows the second suture tail being crossed over the top of the wound in the opposite direction and secured in the opposite cleat on the first clip thus completing the compression crossover step.

FIG. 12 depicts a clip set comprised of two mirror image clips holding an incision line closed.

FIG. 13 is a top view showing the cross over sutures locked into the cleats on each clip.

FIG. 14 is a perspective view showing a clip set in an alternate embodiment in which the suture crossovers are anchored with a half hitch knot around the double cleats.

FIG. 14A is a top view illustrating the first suture crossover pass from the right hand clip to the left hand clip and then back to the cleat on right hand clip to lock in a half hitch knot.

FIG. 14B shows an alternative in which the second suture crossover passes from the left hand clip to the right hand clip and then back to the cleat on the left hand clip where it is locked in a half hitch knot.

FIG. 15 is a top view of an alternate embodiment with the double cleat located peripherally on opposite sides of a pinch groove.

FIG. 16 is a top perspective view showing a clip set similar to that in FIG. 14 with respective single cleats used to secure the crossover suture lines.

FIG. 17 is a top perspective view showing three sets of clips secured along an incision line.

FIG. 18 is a top view of a clip set using a clip much like that shown in FIG. 15, however, an elastic band is shown holding the top edges of the clip together instead of the crossover sutures shown in FIG. 14.

FIG. 19 is a top perspective view showing a clip set like that shown in FIG. 18 but without the integral spring tensioning elements on each clip.

FIG. 20 is a cross sectional view taken along line 20-20 of FIG. 22 showing the tip of a short tooth on one of the clips adapted to penetrate the surface or epithelial skin layer.

FIG. 21 is a cross sectional view taken along line 21-21 of FIG. 22 showing the tip of a deep penetrating tooth.

FIG. 22 is a perspective view of a skin engaging clip with alternating surface or short teeth and deep penetrating or long teeth.

FIG. 23 shows a cross-section through a wound with the first skin tensioning clip engaged in both a deep penetrating area and an upper skin gathering point.

FIG. 24 shows the cross-section through the wound of FIG. 23 with the pair of clips in place and the tissue compressed by the two sets of teeth.

FIGS. 25 and 26 are side and perspective views illustrating a first step of the chemical etching process used to form the tooth profiles of the clips.

FIGS. 27 and 28 are side and perspective views illustrating a second step of the etching process used to form the tooth profiles of the clips.

### DESCRIPTION OF THE DRAWINGS

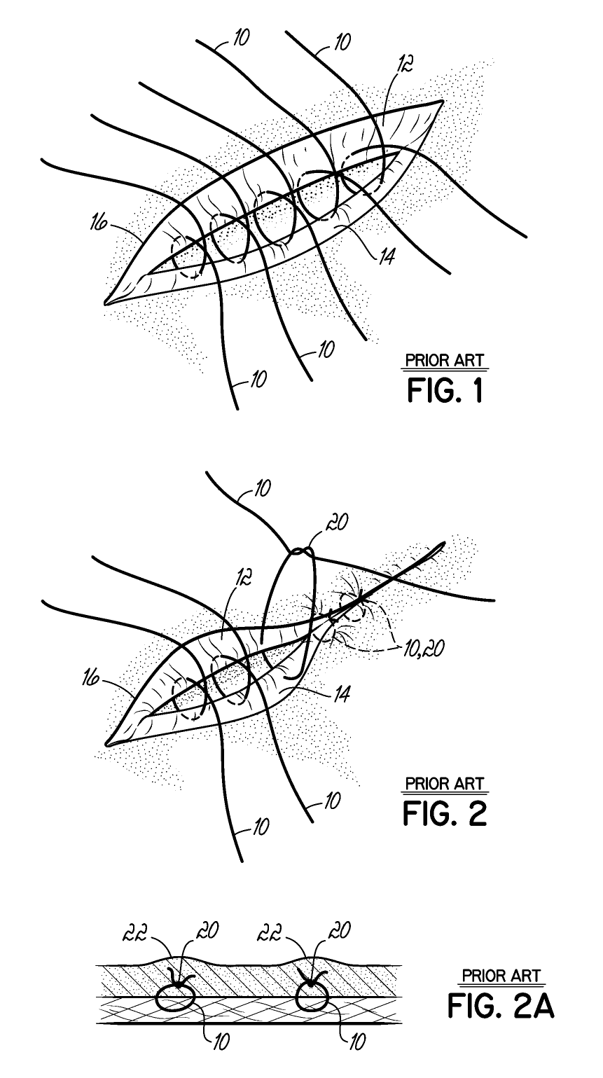

Like reference numerals in the various figures refer to like elements of structure and function. FIGS. 1 and 2 depict the preparation of the prior art layered closure technique for wound closure. Subcutaneous closure stitches 10 are placed through opposite walls 12, 14 of the incision 16. Because the second pass of the needle (not shown) is performed essentially backwards (from the base out to the skin surface), it is very difficult to place this needle if the wound 16 is almost all the way closed, and the wound begins to close as each suture 10 is tightened. Therefore, all sutures 10 which will be required for the subcutaneous closure are placed initially and left loose and untied while access to the area is optimal. Subsequently, the surgeon or other doctor ties each of the sutures 10, pulling the base layers of the wound 16 into close approximation. FIG. 2 shows two of the five sutures 10 tightened and knotted. A third knot 20 is in the process of being tied. The knots 20 are pushed down into the wound 16 below the dermis. Once the top layer of skin is approximated with surface sutures (not shown) the base layers will not be visible. Subcutaneous sutures 10 are almost always made from an absorbable material. Depending on how long it takes for this material to resorb into the tissue, the patient will be able to feel a substantial knot or bump 22 below the surface of the healed wound (see FIG. 2A). Furthermore, during the process of absorbing the foreign material of the suture, the body walls off the foreign material in an effort to break it down. In most cases it eventually goes away, but the byproducts of the foreign body invasion can leave lasting lumps below the surface of the skin.

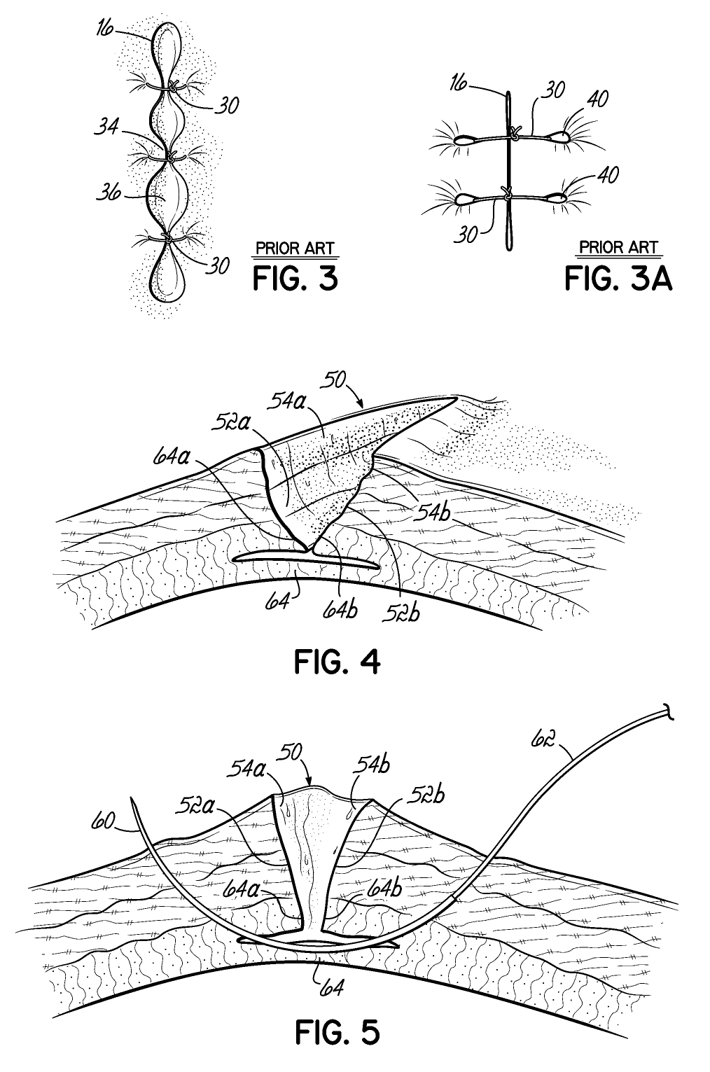

As shown in FIGS. 3 and 3A, to complete the wound closure, surface stitches 30 are used to approximate the epithelial layers of the skin 32. Depending on the technique and the skills of the surgeon, running sutures or an interrupted suture placement technique is used. Quite often, the surface sutures 30 are over-tightened to make sure that the wound does not open up as the swelling (due to the trauma) decreases. This can lead to a wound closure which may be overly tight in some areas 34 and too loose in other areas 36 as schematically shown in FIG. 3. To combat this problem most surgeons put in many more sutures than are typically required to simply approximate the skin.

FIG. 3A depicts the stretched holes 40 which can result at the suture exit site as a result of the tight sutures applied with the prior art technique. This can result in a less than desirable cosmetic outcome.

FIG. 4 depicts a cross-section, in perspective, through a typical wound or incision 50 in tissue, including subcutaneous walls 52a, 52b and epithelial or surface walls 54a, 54b.

FIG. 5 shows the first step in using a system in accordance with an illustrative embodiment of the invention. A needle 60 and suture 62 are passed from the first side of the incision or wound 50 through the deep base 64 and out through the second side of the incision or wound 50. Here, because it may later be easily removed, a monofilament suture 62 may be used in contrast to an absorbable suture.

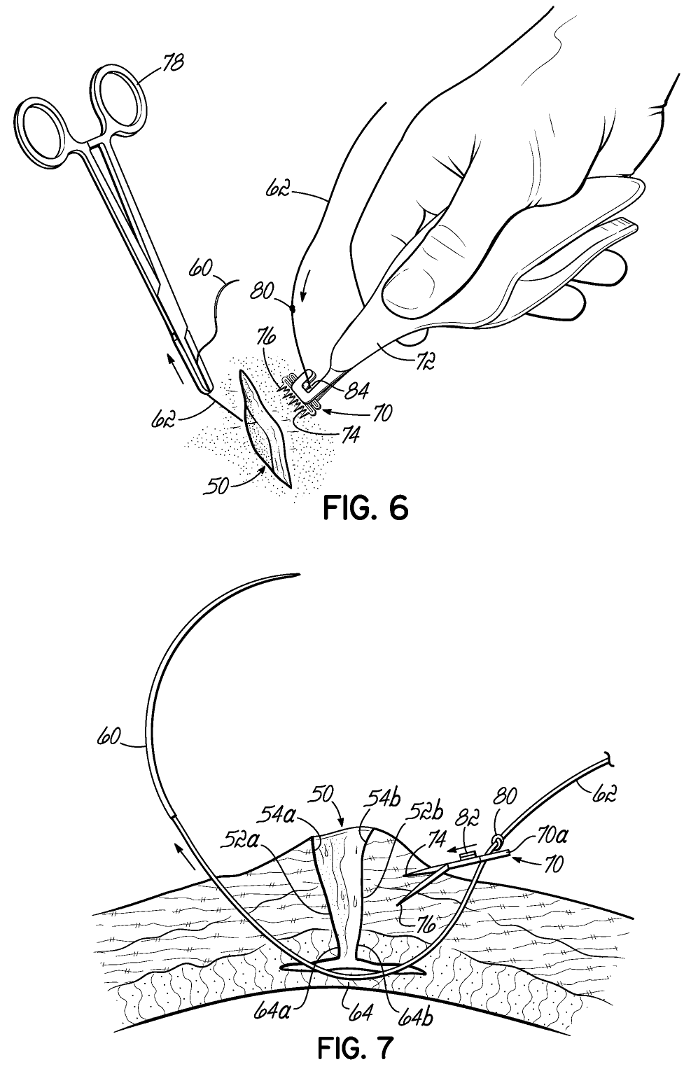

FIG. 6 shows the second step in using the system. A clip 70 having a clip body 70a is positioned by a tool 72. The doctor applies pressure to set the sharp teeth 74, 76 of the clip 70 into the skin. A tool such as a hemostat 78 is used on the opposite side of the wound 50 to pull the suture 62 through the wound 50. An initial locking knot 80 of the suture 62 is set against a connecting portion such as a cleat element 82 (FIG. 7). Knot 80 is shown just prior to being locked into the clip stop element 82. Alternatively, the suture 62 can be positioned into a pinch groove 84 (FIG. 6) to eliminate the need for a knot 80.

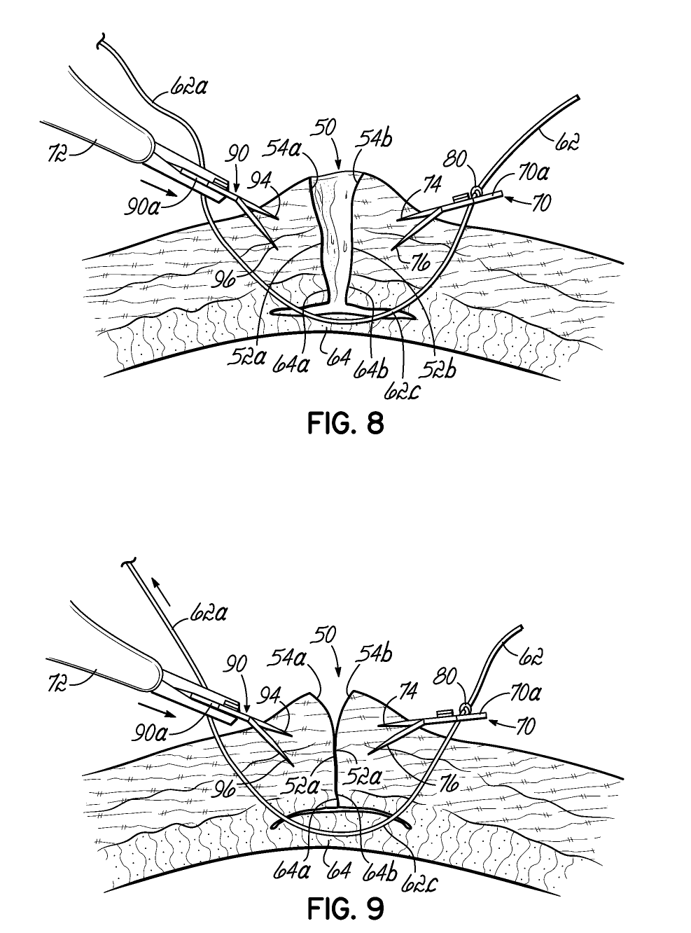

FIG. 7 shows a cross-section of FIG. 6 in which the suture 62 has been locked into the first clip 70. As shown in FIG. 8a second clip 90 having a clip body 90a with teeth 94, 96 extending therefrom is positioned on the opposite side of the wound 50 with the clip placement and tensioning tool 72. By pulling on the loose side of the suture 62 and simultaneously pushing the clip set 70, 90 together with the tool 72 as seen in FIG. 9, the base 64 of the wound 50 compresses as the clips 70, 90 penetrate into the skin and bring the two base edges 64a, 64b into approximation. A portion 62c of the suture 62 extends across the base 64 of the wound 50. At this point, suture end 62a is locked into a pinch groove (not shown) of the second clip 90.

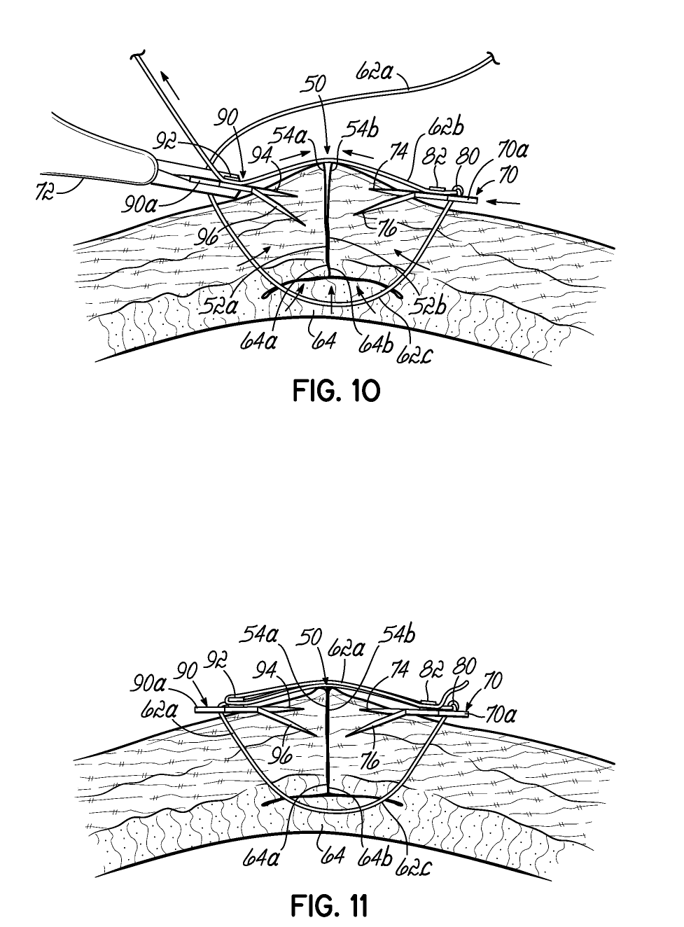

FIG. 10 shows the next step, which is to cross over the first suture tail 62b to the second clip 90 and secure it to a suitable connecting portion, such as cleat 92. This holds the top surface edges of the wound 50 together. As shown in FIG. 11 suture tail 62a is then crossed over in the same manner to the first clip 70 and secured in cleat 93 to complete the approximation. This step closes or approximates the wound edges 52a, 52b and 54a, 54b, compressing the walls of the wound pocket. The final step allows the surgeon to carefully apply the correct compression across the wound edges, everting the skin for a perfect closure. Eversion of the top skin layers is the turning back of the top epithelial layers against each other. This produces the most cosmetically acceptable scar.

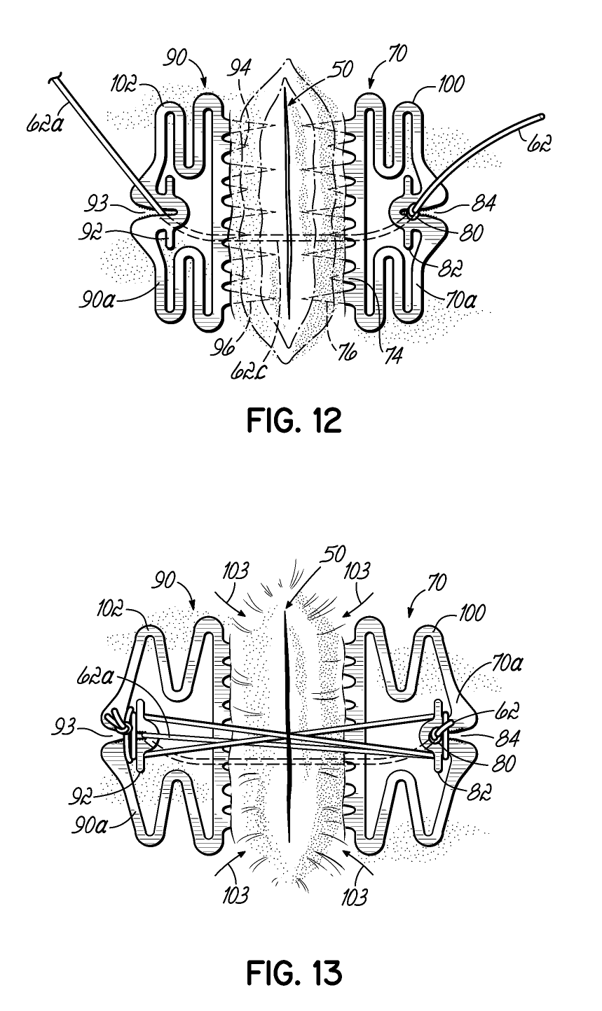

FIG. 12 more specifically illustrates the clips 70, 90 each including an integral tension element or spring 100, 102 integrated into the respective clip body 70a, 90a. These springs 100, 102 are compressed during the suture tensioning step as shown in FIG. 12. More specifically, this figure illustrates the tensioning elements or springs 100, 102 compressed with the suture 62 set into its final position prior to being crossed over and locked down. The compression of the spring elements 100, 102 takes place generally in the direction extending across the wound 50, i.e., in the direction of tension in the suture 62. It will be appreciated that such spring elements may take many integrated or non-integrated forms and may be designed to generate various amounts of spring bias or resilience depending on application needs.

As the traumatic swelling and edema of the wound 50 is resolved, the spring biasing or tensioning elements 100, 102 will extend and thereby pinch the wound edges 54a, 54b (FIG. 11) together in the direction of arrows 103, as shown in FIG. 13. The teeth 74, 76, 94, 96 (FIG. 12) on the clips 70, 90 are continuously and uniformly being urged by the suture 62 and spring bias toward the incision line 98, keeping the wound edges 54a, 54b tightly approximated even as the edema or swelling resolves around the wound 50. FIG. 13 also depicts the crossed over suture 62 tied to the outboard side of the spring elements 100, 102 and secured to the cleats 82, 92 and pinch grooves 84, 93, respectively, on each clip 70, 90. There are many different alternatives that may be used for accomplishing the crossover of the suture 62 and the securing of the same.

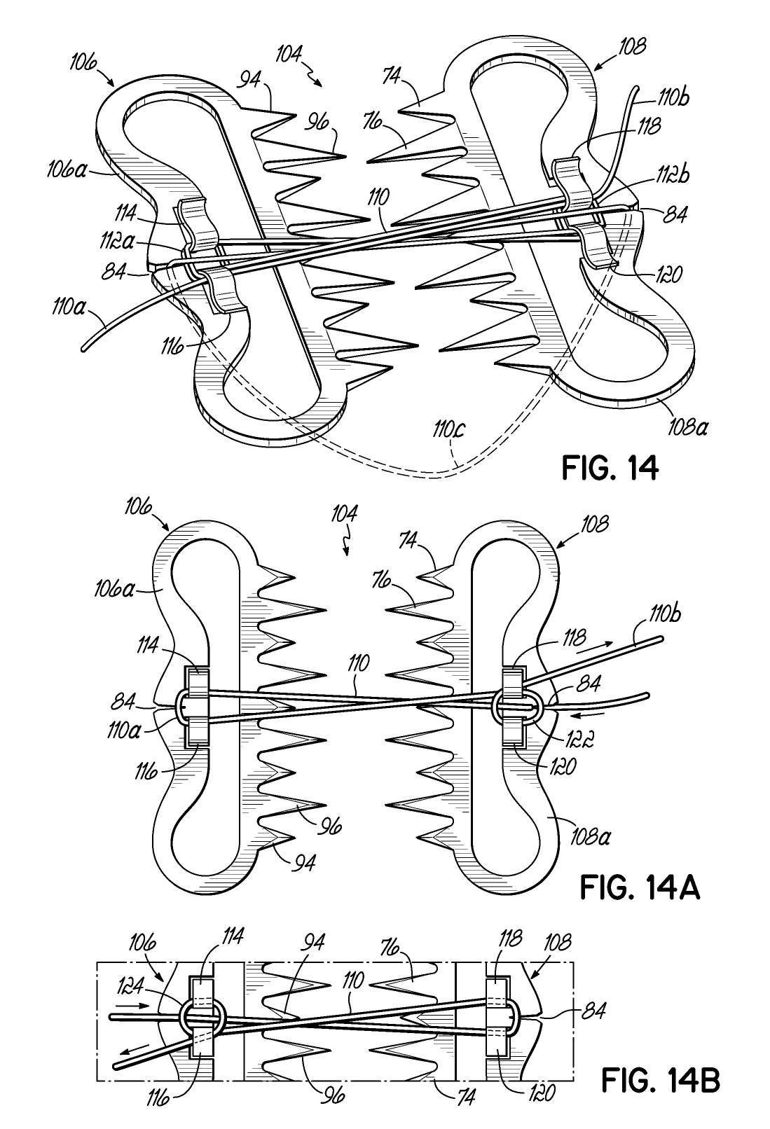

FIG. 14 illustrates an embodiment of a clip system 104 with first and second clips 106, 108 each having a clip body 106a, 108a and a suture 110 in which suture tails 110a, 110b have crossed over and locked with half hitch knots 112a, 112b under double cleats 114, 116 and 118, 120 on each clip 106, 108. Another portion 110c of suture 110 extends through a base of the wound (not shown).

In another alternative, FIG. 14A illustrates the first pass of the suture cross from clip 106 to clip 108 passing under double cleat 114, 116 ending with a locking half hitch knot 122 under double cleat 118, 120 on clip 108. FIG. 14B illustrates another alternative involving a cross over pass of the suture tail from clip 108 to clip 106 passing under double cleat 118, 120 and then locking with a half hitch knot 124 on double cleat 114, 116.

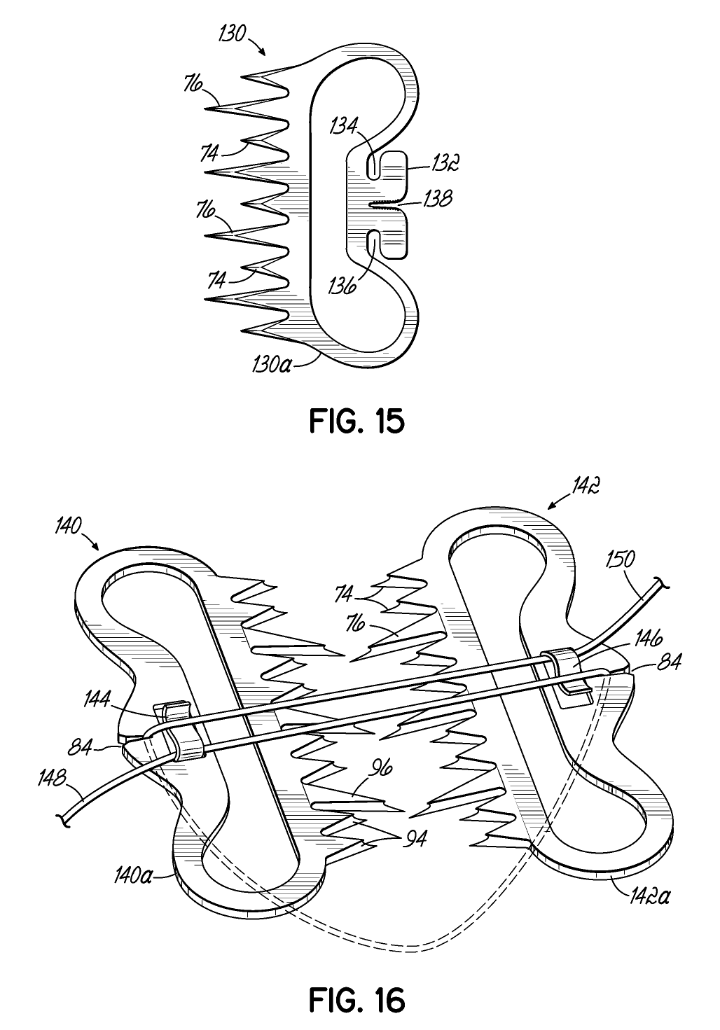

FIG. 15 illustrates an alternative embodiment of a clip 130 with a double cleat 132 having grooves 134, 136 located peripherally on either side of a single pinch groove 138 which grips the suture tail (not shown) exiting the skin.

FIG. 16 illustrates a set of clips 140, 142 each having a clip body 140a, 142a similar to those shown in FIG. 14, but with respective single cleats 144, 146 used to secure the crossover suture tails 148, 150.

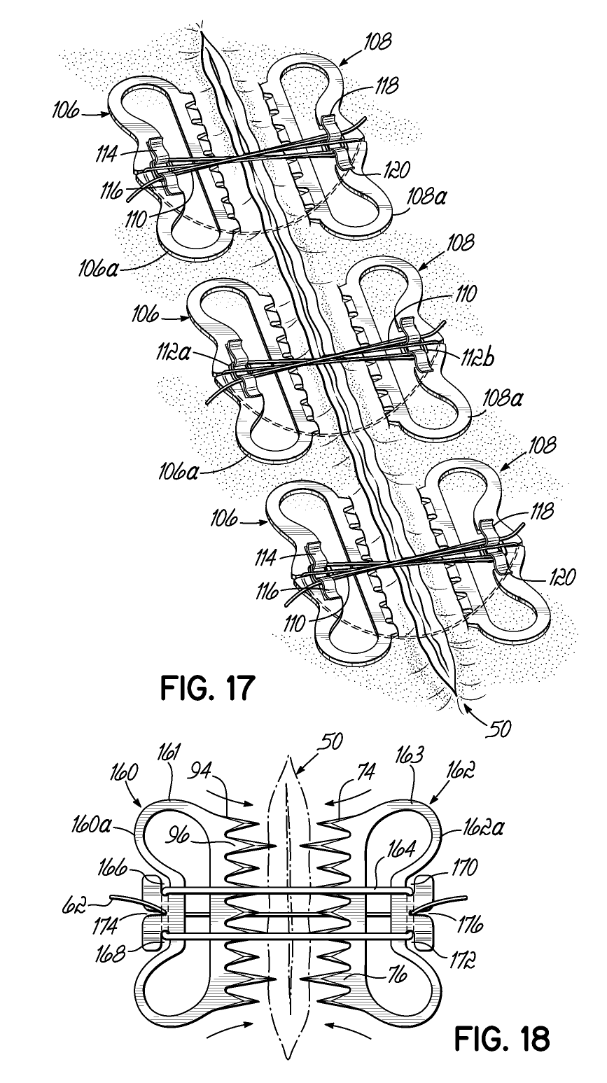

FIG. 17 illustrates three respective sets of suture clips 106, 108 spaced laterally along the wound 50. In this alternative, more than one clip set is used to close a longer incision. Any number of clip sets may be used as needed to close the entire length of the incision or wound line. All of the deep base sutures (i.e., one for each clip set) would be applied and then the individual clip sets would be applied as described above, including tensioning and securing with the respective deep base suture. In this example, three suture clip sets 106, 108 are used in a situation where as many as eight interrupted sutures of the prior art might be required to close a similar incision.

FIG. 18 illustrates a clip set using clips 160, 162 having clip bodies 160a, 162a much like that shown in FIG. 15. In this embodiment, however, a flexible tensile member in the form of an elastic band 164 holds the top edges of the clips 160, 162 together instead of the crossover sutures shown in FIG. 14. The elastic band 164 is retained in respective grooves 166, 168 and 170, 172. This embodiment provides a non-integrated spring biasing or tensioning element 164 for supplying measure of compliance to urge the wound edges together during the reduction of swelling while maintaining a close approximation of the epithelial layers throughout the healing process. The clip bodies 160a, 162a also have integrated spring biasing elements 161, 163 for supplying resilience during the healing process as previously discussed. The elastic band 164 in this embodiment serves a dual purpose as it provides resilience and acts as a flexible tensile member similar to a suture. A deep base suture 62 is used as previously described and is locked into respective pinch grooves 174, 176.

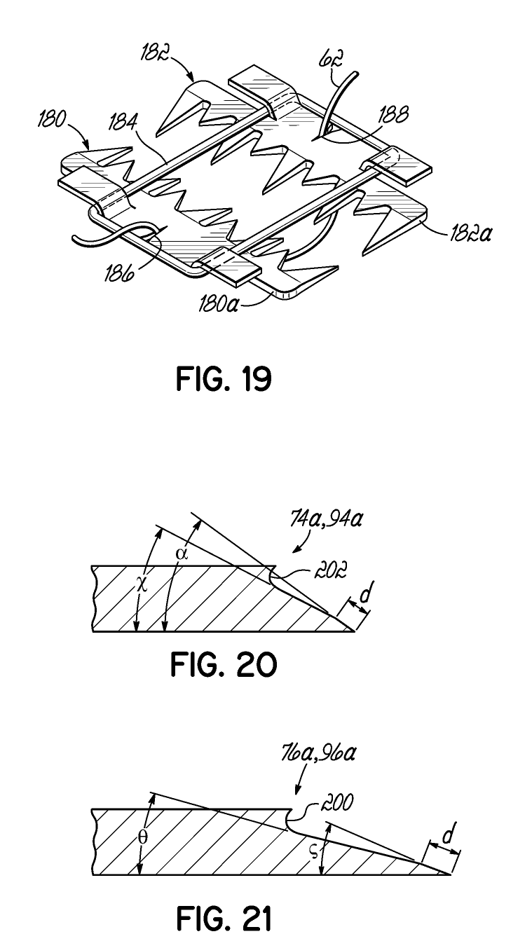

FIG. 19 illustrates a set of clips 180, 182 with clip bodies 180a, 182a not having spring elements. In this embodiment, an elastic holding band 184 supplies the automatically adjustable tensioning and compliance to hold the skin clips 180, 182 closed during the wound healing period. A deep base suture 62 is used as previously described and is locked into respective pinch grooves 186, 188.

FIGS. 20 and 21 respectively illustrate the different cross sectional designs of the tooth tip configurations. These two different tooth profiles or configurations are combined and alternated on the clip to perform separate functions as discussed. The teeth tips 74a, 94a and 76a, 96a have dual angle cutting edges. The respective angles α and ζ closest to the tip are steeper than the angles χ and θ farther from the tip. The steeper and shorter profile of the tooth tip 74a, 94a shown in FIG. 20 is used at the top edges of the wound 50 to initially penetrate and gather the surface of the skin. The profile of the tooth tip 76a, 96a illustrated in FIG. 21 includes a shallower second angle θ that creates a longer initial penetrating tip. This profile allows the longer teeth 76, 96 to penetrate deeper and slightly underneath the shorter teeth 74, 94 to thereby compress the wound pocket below the surface. The longer, extended tooth profile shown in FIG. 21 is bent or angled downward from the overall or main plane of the clip 70 or 90 to penetrate deeper and close to the pocket of the wound. See FIGS. 10 and 11, which illustrate this feature. The sharp penetrating tip extends for a short length “d” at the tips 74a, 76a, 94a, 96a. The second angles χ and θ rise more gradually at a smaller angle and stop at respective sharp undercuts 200, 202 resulting in compression stop elements.

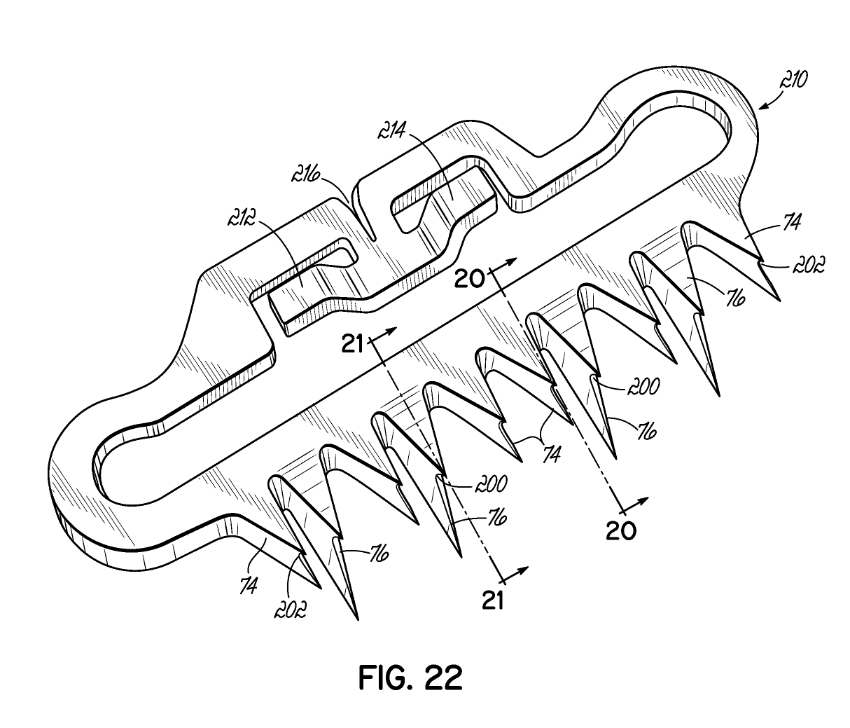

FIG. 22 illustrates a clip 210 with a clip body 210a and alternating tooth profiles as may be used in any of the embodiments. An integrated double cleat 212, 214 and pinch groove 216 is incorporated for previously described purposes related to the securement of one or more flexible tensile members.

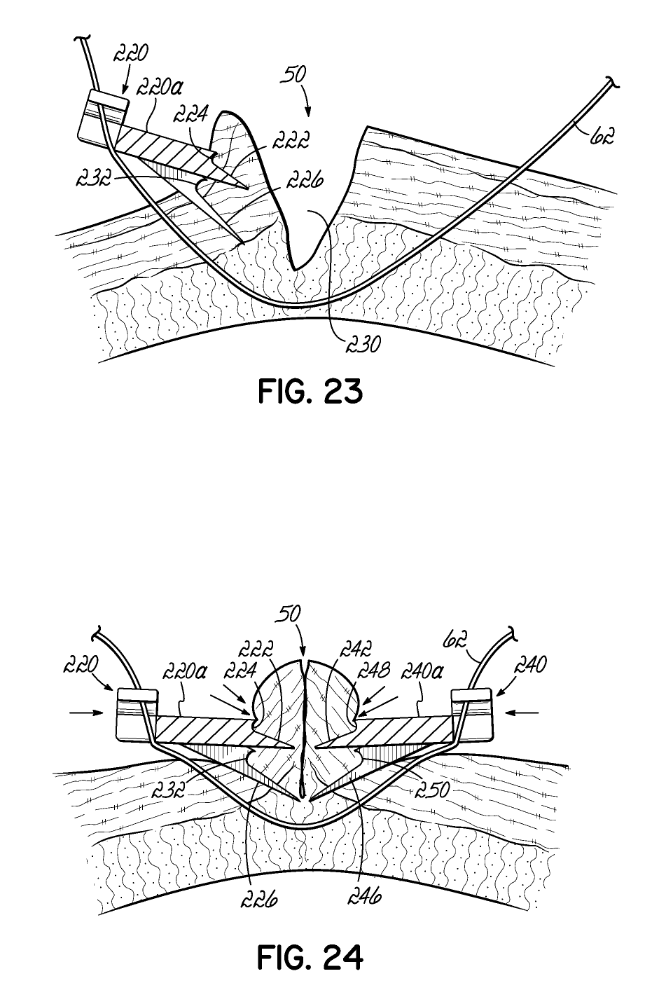

FIGS. 23 and 24 illustrate a cross section of a wound 50 into which a deep base suture 62 has been applied. In FIG. 23, one side of a clip 220 with a clip body 220a is initially applied to the edge of the wound 50. Surface teeth 222 (one shown) penetrate near the top of the skin and are restricted from further penetration by the compression stop element 224. The deeper penetrating teeth 226 (one shown) protrude farther into the tissue and force the wound pocket 230 to close. The longer, deep penetrating teeth 226 are also restricted from further penetration by compression tips or stop elements 232. In FIG. 24, a second clip 240 with a clip body 240a and respective teeth 242, 246 is shown. The first clip 220 and second clip 240 are shown as compressed toward each other and close the wound 50. The compressive force applied with the compression tips or stop elements 224, 232, 248, 250 of the teeth 222, 226, 242, 246 forces the surface skin to be gathered up and also approximated. The surface teeth 222, 242 act to bring the surface tissue together and the deeper teeth 226, 246 compress the deeper wound pocket area.

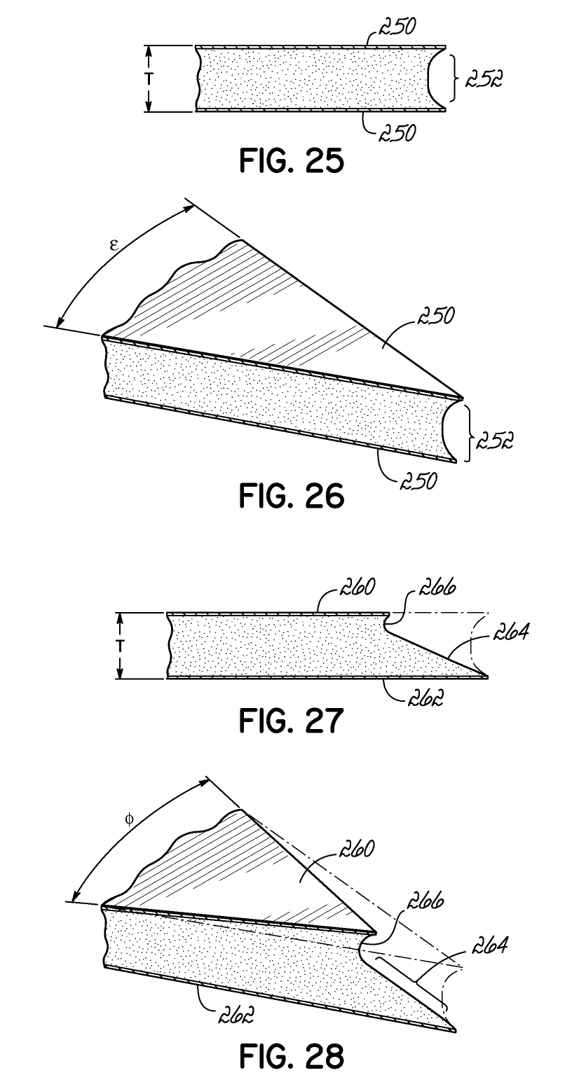

In another alternative aspect of the invention, a manufacturing technique for the teeth on the clips is illustrated in FIGS. 25-28. An economical manner of creating shapes from flat metal stock is to chemically etch the stock in a process referred to as photo fabrication. Photo fabrication is a process similar to that used to create printed circuit boards. Flat sheets of steel are coated on one or both sides with a photosensitive resist. A photographic negative is placed on top of the photo resist and the resist is exposed to light. The photo resist area that is exposed to light changes and becomes hardened. The negative is removed and the resist is treated to remove the unhardened material. In the area in which the resist is removed, bare metal is exposed. The entire metal sheet is treated in a caustic acid bath which removes the exposed bare metal portions by chemical etching. Some or all of the metal sheet thickness is removed during the etch process. This manufacturing method is used to make intricate metal parts that would be otherwise difficult to machine or stamp. One of the advantages of using this process is the ability to obtain sharp features without the burrs often left behind during stamping. If done carefully, this process is also capable of etching sharp points without secondary grinding processes.

In order to create the stepped tooth profile described in connection with FIGS. 20 and 21, a multi-step process is provided. A metal plate such as stainless steel or other biocompatible metal is initially etched to have teeth with a tooth angle ε. In this first step, as depicted in FIGS. 25 and 26, a typical chemical etching process is used and includes a hardened photo resist 250 with the same negative used on the top and bottom of the steel sheet stock. After etching, the etched steel exhibits a signature artifact, i.e., the undermining of a stainless steel towards the center section 252 of the thickness of the metal sheet. This artifact is the result of the way sheet steel is rolled to thickness. By compressive rolling, the mill hardens the top and bottom but leaves the grain structure of the steel in the middle of the cross section comparatively soft or more porous. The chemical etching is more efficient on the softer core or middle section of the cross section or thickness “T” and, therefore, this section is etched faster than the two hardened outer surfaces. The undermining is a typical byproduct of this type of manufacturing and is usually considered to be a negative artifact because of the razor-sharp edges it leaves at the top and bottom of the part. However, this may be used to advantage in the formation of the teeth of the clip.

In a second step, another layer of photo resist is applied on one side of the part and etching is performed a second time with a different photo negative. FIGS. 27 and 28 illustrate the second etching process in which a new shape of resist 260 is hardened with the second negative on the top surface of the tooth profile. This may be shorter and more steeply angled (see angle φ) than photoresist 250. A photo resist 262 is also placed on the bottom profile to prevent erosion of the sharp point obtained in the first etching process. Alternatively, the photo resist 262 may be slightly modified to enhance the sharpness in the second etching process. As in the first etching process, the second etching process undermines farther back from the sharp penetrating point in an area 264 of the tooth profile. This creates an elongated, sharp penetrating tip with a sharp compression point or stop element 266 on the top surface. This tooth profile provides the ability of the tooth to penetrate and engage the skin up to a predetermined depth after which penetration is stopped at the compression tip or stop element 266. When these tooth profiles are configured in a line as on a skin engaging clip, continued inward pressure gathers and compresses the wound edges to create a wound approximating line that heals effectively and in cosmetically desirable manner.

The chemical etching process may be varied to create sharper and/or longer penetrating tips in order to penetrate deeper or shallower as necessary to accommodate different tissue types or clip sizes. In addition, the included angle ε (FIG. 26) etched in the first step may be the same as or different than the included angle φ of the sharpening step. The clips are typically formed in asymmetric fashion so that tooth profiles on opposite sides of the wounds tend to interdigitate or nest between each other. If the teeth are spaced too closely together, tissue tends to necrose and not heal well. Necrosis stems from over compression of the capillary bed of the skin. If the tooth spacing is proper, a light pink cast of skin will prevail in the first six days of healing and this signals proper capillary flow while still approximating the wound and allowing for collagen buildup and wound healing. If the tooth spacing is too far apart, gaps are left between the teeth where bacterial can collect and infection may set in. This may also lead to excessive scar tissue.

The devices shown and described herein may be used in any situation where the closure of a wound or incision is required. Examples outlined in this disclosure are directed towards the removal of questionable lesions and the closure of the opening or wound produced as a result. These examples should not be construed as limiting the use of the devices shown and described herein. For example, the device or system disclosed herein may be used for large trauma wounds which need layered closure, or for diagnostic procedures such as Mohs surgery. The devices and methods disclosed herein are specially well suited for reattachment of flaps or skin grafts. While this invention is not limited to removal of skin lesions, this represents one of the largest volume procedures requiring acute wound closure.

While the present invention has been illustrated by a description of various preferred embodiments and while these embodiments have been described in some detail, it is not the intention of the Applicants to restrict or in any way limit the scope of the appended claims to such detail. Additional advantages and modifications will readily appear to those skilled in the art. The various features discussed herein may be used alone or in any combination depending on the needs and preferences of the user. This has been a description of illustrative aspects and embodiments the present invention, along with the preferred methods of practicing the present invention as currently known. However, the invention itself should only be defined by the appended claims. What is claimed is:

1. A wound closing clip, comprising:
a clip body;
a first plurality of teeth extending in a first direction from the clip body for engaging superficial skin tissue;
a second plurality of teeth extending in a second direction from the clip body for engaging tissue below the superficial tissue, the second direction being different than the first direction and configured to direct the second plurality of teeth into deeper tissue than the first plurality of teeth; and
a flexible tensile member connecting portion coupled with said first and second pluralities of teeth, said flexible tensile member connecting portion including an element capable of securing a flexible tensile member extending below the tissue engaged with the second plurality of teeth and across a base of the wound.

2. The wound closing clip of claim 1, wherein the clip body further comprises a spring element positioned generally between the connecting portion and the first and second pluralities of teeth for providing resilience generally in the first direction.

3. The wound closing clip of claim 1, wherein said flexible tensile member connecting portion further comprises at least one of a notch and/or a cleat.

4. The wound closing clip of claim 1, wherein at least one of the first and second pluralities of teeth comprise teeth with sharp points extending from respective compression stop elements for engaging tissue and limiting the depth that the sharp points penetrate into the tissue.

5. The wound closing clip of claim 1, wherein the clip body is generally planar and the second direction is a downward direction out of a plane containing the clip body.

6. A system for closing a wound, comprising a pair of clips and a flexible tensile member, each of said clips comprising:
a clip body;
a first plurality of teeth extending in a first direction from the clip body for engaging superficial skin tissue;
a second plurality of teeth extending in a second direction from the clip body for engaging tissue below the superficial skin tissue, the second direction being different than the first direction and configured to direct the second plurality of teeth into deeper tissue than the first plurality of teeth; and
a flexible tensile member connecting portion coupled with said first and second pluralities of teeth, said flexible tensile member connecting portion including an element capable of securing a flexible tensile member extending below the tissue engaged with the second plurality of teeth and across a base of the wound;
wherein the first and second pluralities of teeth of the respective clips are adapted to be driven into tissue on opposite sides of the wound, and the flexible tensile member is directed through the tissue and beneath each of the second pluralities of teeth and coupled to the flexible tensile member connecting portions of the respective clips, thereby approximating edges of the wound.

7. The system of claim 6, further comprising a spring tensioning element including at least one of: 1) a spring element integrated into at least one of the clip bodies generally between the connecting portion and the first and second pluralities of teeth for providing resilience generally in the first direction, or 2) a separate spring tensioning element coupled between the pair of clips.

8. The system of claim 6, wherein said flexible tensile member connecting portion further comprises at least one of a notch and/or a cleat.

9. The system of claim 6, wherein at least one of the first and second pluralities of teeth comprise teeth with sharp points extending from respective compression stop elements for engaging tissue and limiting the depth that the sharp points penetrate into the tissue.

10. The system of claim 6, wherein the clip body is generally planar and the second direction is a downward direction out of a plane containing the clip body.

11. A wound closing clip, comprising:
a clip body including first and second opposite sides;
a plurality of teeth extending from the first side for engaging skin tissue;
a flexible tensile member connecting portion on the second side, said flexible tensile member connecting portion including an element capable of securing a flexible tensile member extending below the tissue engaged with the plurality of teeth and across a base of the wound; and
a spring element incorporated into the clip body and positioned generally between the plurality of teeth and the connecting portion for providing resilience generally in the direction that the first plurality of teeth extend from the first side.

12. A method of closing a wound using first and second clips and a flexible tensile member, each of the clips comprising a first plurality of teeth extending in a first direction for engaging superficial skin tissue, a second plurality of teeth extending in a second direction different than the first direction for engaging tissue below the superficial skin tissue, and a flexible tensile member connecting portion coupled with said first and second pluralities of teeth, the method comprising:
driving the first and second pluralities of teeth of the first clip respectively into the superficial skin tissue and into the tissue below the superficial skin tissue on one side of the wound;
driving the first and second pluralities of teeth of the second clip respectively into the superficial skin tissue and into the tissue below the superficial skin tissue on an opposite side of the wound;
directing the flexible tensile member below the second pluralities of teeth and across a base of the wound;
tensioning the flexible tensile member to pull the first and second clips toward one another and approximate edges of the wound; and
coupling the flexible tensile member to the respective flexible tensile member connecting portions.

13. The method of claim 12, comprising:
resiliently compressing the first and second clips generally in directions toward the wound.

14. The method of claim 12, wherein coupling the flexible tensile member to the respective flexible tensile member connecting portions further comprises coupling the flexible tensile member to a notch and/or a cleat.

15. The method of claim 12, wherein driving the first and second pluralities of teeth respectively into the superficial skin tissue and into the tissue below the superficial skin tissue further comprises:
engaging the tissue with sharp points and compression stop elements, the stop elements limiting the depth of penetration of the sharp points and forcing the tissue into approximation, the sharp points extending from the compression stop elements.

16. The method of claim 12, further comprising:
connecting the first and second clips together in tension above the wound to approximate superficial skin tissue on opposite sides of the wound.

17. A method of closing a wound using first and second clips and a flexible tensile member, each of the clips comprising a clip body including first and second opposite sides, a plurality of teeth extending from the first side for engaging skin tissue, a flexible tensile member connecting portion on the second side, and a spring element incorporated into the clip body and positioned generally between the plurality of teeth and the connecting portion, the method comprising:
driving the plurality of teeth of the first clip into the skin tissue on one side of the wound;
driving the plurality of teeth of the second clip into the skin tissue on an opposite side of the wound;
directing the flexible tensile member below the teeth of the first and second clips and across a base of the wound;
tensioning the flexible tensile member to pull the first and second clips toward one another and approximate edges of the wound;
coupling the flexible tensile member to the respective flexible tensile member connecting portions; and
resiliently compressing the spring elements of the first and second clips generally in directions toward the wound.

18. The method of claim 17, further comprising:
connecting the first and second clips together in tension above the wound to approximate superficial skin tissue on opposite sides of the wound.

19. The method of claim 17, further comprising:
allowing the spring elements to expand in directions away from the wound as the wound heals.

20. A method of manufacturing a wound closing clip comprised of a clip body and a plurality of tissue engaging teeth extending from the clip body, the method comprising:
forming the clip body with a plurality of tissue engaging teeth having converging points,
chemically etching the converging points in a first chemical etching process to form sharp upper and lower edges of the converging points, and
chemically etching the converging points in a second chemical etching process to form a compression stop element located inward along the length of each tooth from the converging point.
